(19) United States
(12) Patent Application Publication
(10) Pub. No.: US 2024/0229117 A1
(43) Pub. Date: Jul. 11, 2024

(54) MULTIPLEXED PROTEIN AND NUCLEIC ACID DETECTION METHOD WITH ROLLING CIRCLE AMPLIFICATION (RCA) ASSISTED COLOR COMBINATORIAL AND SEQUENTIAL FLUORESCENCE IN SITU HYBRIDIZATION (CCS-FISH)

(71) Applicant: The Hong Kong University of Science and Technology, Hong Kong (CN)

(72) Inventors: Shuhuai YAO, Hong Kong (CN); Binbin CUI, Hong Kong (CN)

(21) Appl. No.: 18/406,932

(22) Filed: Jan. 8, 2024

Related U.S. Application Data
(60) Provisional application No. 63/478,697, filed on Jan. 6, 2023.

(51) Int. Cl. C12Q 1/6841

(52) U.S. Cl. CPC C12Q 1/6841; C12Q 2600/166

(57) ABSTRACT

Materials and methods for capturing target proteins and analyzing the proteins using an antibody conjugated to an oligonucleotide are provided. Methods of using rolling circle amplification (RCA) to amplify to the oligonucleotide are also provided. The oligonucleotide contains multiple sites for probe binding, which allows for multiplexity by means of color-combinatorial and sequential fluorescence in situ hybridization (ccs-FISH).

### CROSS-REFERENCE TO RELATED APPLICATION

This application claims the benefit of U.S. Provisional Application Ser. No. 63/478,697, filed Jan. 6, 2023, which is hereby incorporated by reference in its entirety including any tables, figures, or drawings.

### REFERENCE TO SEQUENCE LISTING

The Sequence Listing for this application is labeled “HKUS186X.xml” which was created on Dec. 19, 2023 and is 2,847 bytes. The entire content of the Sequence Listing is incorporated herein by reference in its entirety.

### BACKGROUND OF THE INVENTION

Biologists have long been seeking to delineate the tissue construction in terms of not only the cellular compositions but also the spatial interactions among each type of cells. A hierarchical study of the cellular state, the cellular composition, and the spatial organization of different types of cells can play an essential role in understanding embryonic development, tissue homeostasis, disease origination and progression, and personalized treatment. Spatial protein profiling is used to exploit the 3-dimensional (3D) architecture of the tissue. For instance, protein staining is used to visualize the cellular components involved and structural variation in disease progression. Multiplexed spatial protein profiling has been validated to portray the microenvironment of tumors and autoimmune diseases, such as the lupus.

The delineation of the complex tissue context in situ requires high-dimensional protein profiling. Currently, the detection of the proteins in situ relies on antibodies. Though a multiplexity of greater than 50 markers have been achieved by the application of cyclic immunofluorescence, these technologies suffer from assorted drawbacks. The repetitive antibody removal and incubation, such as in multi-epitope-ligand cartography, cyclic immunofluorescence, multiplexed fluorescence microscopy, and iterative bleaching extends multiplexity, is time-consuming and labor intensive, lasting weeks to complete the protein profiling of an individual sample. Co-detection by indexing circumvents this issue with more kinetic oligo chemistry; however, due to the lack of signal amplification, it is rarely applied to tissues with high autofluorescence. Immunostaining with signal amplification by exchange reaction equips the oligo-antibody conjugate with progressive amplification through DNA branching. Nonetheless, the involvement of long DNA concatemers will complicate the reaction when high multiplexity is required in the experiment and weaken the penetration efficiency into the deep tissue. Moreover, all of these methods use a single color to represent an individual protein target, which generates a massive amount of data, leading to cumbersome data processing. The non-fluorescence protein profiling, such as the imaging mass cytometry, multiplexed ion beam imaging, and multiplexed vibrational imaging, obtain the multiplexed image via scanning the sample point by point, rendering the image acquisition of large samples exceptionally slow. Due to the fastidious requirements of the specialized equipment and expertise for the operation, these technologies are inaccessible to most laboratories.

Therefore, it is essential to devise a novel method that fulfills the following requirements for the protein detection: high multiplexity achievable to cover all of the target proteins of interest for individual research; all-in-one staining, which incubates all target proteins with a cocktail of antibodies; signal amplification compatible with different tissue preparations and auto-fluorescent tissues; experimental simplicity with minimum cycles; and a high resolution but low hard disk memory consumption.

### BRIEF SUMMARY OF THE INVENTION

Certain embodiments of the invention provide materials and methods for capturing target proteins and optionally, further analyzing the proteins, including using an antibody conjugated to an oligonucleotide to label the protein. The subject invention further pertains to methods using rolling circle amplification (RCA) to amplify the oligonucleotide. The oligonucleotide can contain a certain number of sites for probe binding, which can allow for multiplexity by means of color-combinatorial and sequential fluorescence in situ hybridization (ccs-FISH).

In certain embodiments, a DNA oligonucleotide primer sequence is conjugated to an antibody, followed by the incubation of the antibody cocktail with a sample (e.g., a tissue). In certain embodiments, after removing unbound antibodies, at least two different types of DNA sequences: a circular single stranded DNA sequence or a linear DNA padlock probe can be added to the sample, in which each circular single stranded DNA sequence or linear padlock DNA probe can specifically bind to a type of oligonucleotide primer conjugated to the antibody. In certain embodiments, a ligation step can then occur to circularize each single stranded DNA padlock probe. In certain embodiments, alternatively, a pool of circular DNA can be directly added to the mixture of antibody-oligonucleotide primers bound to the tissue, in which each of circular single stranded DNA is specific to one type of antibody-oligonucleotide conjugate. In certain embodiments, after the direct binding of each circular single stranded DNA sequence to the oligonucleotide primers or ligation of the DNA padlock probes annealed to the oligonucleotide primers, rolling circle amplification (RCA) in situ and ccs-FISH can be performed. In certain embodiments, a DNA polymerase, such as Phi29, can be used to perform an RCA.

In preferred embodiments, about 2 to about 20 different types of FISH probes, each type emitting a unique light color, are used to stain types of protein targets based on the corresponding color combination, such as, each, 2 FISH probes can be used to stain 3 types of proteins, 4 FISH can be used to stain 15 FISH probes, or 8 FISH probes can be used to stain 255 proteins. In certain embodiments, one fluorescent image is taken for each type of FISH probe. In certain embodiments, these images are overlayed in a single image or in a combination of two, three, four or more images, allowing for the recognition of different types of antibodies. After an image is taken, the FISH probes can be removed with a washing buffer, for example, a formamide buffer. In certain embodiments, additional rounds of adding n types of FISH probes and subsequent fluorescent imaging can be performed until all antibodies are imaged.

In preferred embodiments, cyclic ccFISH can be used to read the DNA amplicons of the RCA. In preferred embodiments, four types of FISH probes are used to stain 15 types of protein targets based on their corresponding color combination. In certain embodiments, one fluorescent image is taken for each type of FISH probe, such as, for example, four fluorescent images for each of the four different colors of FISH. In certain embodiments, these images are overlayed in a single image or in a combination of two, three, four or more images, allowing for the recognition of different types of antibodies. After the image is taken, the FISH probes can be removed with a washing buffer, such as, for example, a formamide buffer. In certain embodiments, additional rounds of adding four types of FISH probes and subsequent fluorescent imaging can be performed until all antibodies are imaged.

In certain embodiments, an RNA oligonucleotide is reversely transcribed to yield a DNA oligonucleotide primer. In certain embodiments, the sequence of an RNA oligonucleotide is transferred to DNA oligonucleotide through the RNA templated PBCV-1 DNA Ligase or Chlorella virus DNA Ligase (e.g., SplintR® (New England Biolabs, Waltham, MA)) ligation to yield the DNA oligonucleotide primer. In certain embodiments, a PBCV-1 DNA Ligase or Chlorella virus DNA Ligase can be used to transfer a microRNA sequence to a cDNA sequence immobilized on the glass surface.

In certain embodiments, a rolling circle amplification reaction is initialized by a DNA polymerase, which replicates the circularized padlock probe up to thousands of times. In certain embodiments, the replication of the circularized padlock probe creates a single strand of linear DNA up to tens of thousands bases. The resulting long single-stranded, linear DNA coils to form a DNA tangle with a size of less than about 500 nm in diameter, which can be detected with next generation sequencing, in situ sequencing, or, preferably, fluorescence in situ hybridization (FISH). In certain embodiments, the DNA tangle comprises a tandem repeat complementary to the padlock probe sequence; it can accommodate thousands of copies of binding sites for each probe.

In certain embodiments, the padlock probe comprises two target recognition sites (TRS), each ranging from about 10 to about 15 bases long. In preferred embodiments, each of the two TRS are the same length, such as for example 10 bases long or 15 bases long. In certain embodiments, the padlock probe further comprises an intermediate encoding region that is about 30 to about 230 bases long. In certain embodiments, the intermediate encoding regions comprises about 3 to about 30 binding sites for FISH probes. In certain embodiments, the FISH probes are about 8 to about 15 bases long. In certain embodiments, the padlock probe is about 50 bases long to about 250 bases long. In certain embodiments, the number of FISH probe binding sites in the intermediate coding region is about 3 to about 30 and each TRS can be about 10 to about 15 bases long. In certain embodiments, the number of FISH probe binding sites in the intermediate coding region is 7 and each TRS can be about 10 bases long. In certain embodiments, the two TRS regions can be about 15 bases long each, and the intermediate region can be about 120 bases long, which can accommodate 12 probe binding sites, in which the multiplexity can be expanded to N¹², in which N is the number of colors in each cycle and the conventional number can be 4.

In certain embodiments, the detection of the RCA amplicon of each type of padlock probe can be achieved by color combinatorial FISH (ccFISH), sequential FISH (seq-FISH), cyclic ccFISH, or color combinatorial and sequential FISH (ccsFISH). In certain embodiments, cyclic ccFISH can be used for spatial protein detection.

In certain embodiments, the subject methods further pertain to the detection of microRNA or blood protein, in which either seq-FISH or ccs-FISH can be used to perform the quantification. In certain embodiments, the TRS regions of the padlock probe are 15 bases long, the padlock probe is 90 bases long, and the multiplexity of seq-FISH and ccs-FISH are 4⁶=4096, and 15,448 respectively.

### BRIEF DESCRIPTION OF THE DRAWINGS

FIGS. 1A-1D Overview of the workflow. (FIG. 1A) The padlock probe anneals to the primer sequence in a manner such that the 3′ and 5′ end of the padlock probe are juxtaposed to each other, followed by the circularization catalyzed with T4 ligase. Alternatively, circular DNA can directly bind to the primer sequence. The Phi29 will initialize the rolling circle amplification (RCA) to make up to thousands of DNA repeats complementary to the padlock probe. The single stranded DNA synthesized will coil up in solution to become a DNA tangle that can be detected with fluorescence in situ hybridization. (FIG. 1B) High multiplexity can be accomplished with color combinatorial FISH (ccFISH), in which a DNA tangle can be stained either with one color or a combination of colors from n available colors in a single cycle. The multiplexity can be 2ⁿ-1, where n is the number of colors available in a single cycle. (FIG. 1C) Massive multiplexity can be achieved through sequential FISH. Since each padlock probe possesses a certain number of probe sites, corresponding to a barcode, the DNA tangle will contain up to thousands of copies of binding sites for each probe. All DNA tangles are stained in binding-site by binding-site fashion, in which one of the probe binding sites is detected in every cycle and after imaging the FISH probes are stripped off with formamide buffer. Therefore, every DNA tangle will generate a barcode after a certain number of imaging cycles, equivalent to a multiplexity of n″, where n is the number of colors available in each cycle and m is the number of cycles. (FIG. 1D) The detection throughput of the seq-FISH can be expanded with the color combinatorial FISH (cc-FISH). All DNA tangles are stained with n types of FISH probes of different colors, where n is the number of colors available in a single cycle. In a single cycle, a multiplexity of 2^{m}-1 can be achieved. In the ccFISH, a DNA tangle stained with k colors (k≤n) will consume k binding sites for FISH probe. In the next cycle, the ccFISH is performed over the remaining FISH probe binding sites. The number of the multiplexity can be calculated with exhaustive enumeration. For example, when 4 colors are used to encode the DNA tangles possessing 6 probe binding sites, with each DNA tangle stained with only one or two colors, the multiplexity can be 15,448.

FIGS. 2A-2B Schematics of RCA-assisted cyclic cc-FISH applied to spatial protein profiling.

FIG. 3 Schematic of the RCA-assisted ccs-FISH applied to the protein profiling of the blood plasma.

FIGS. 4A-4B. FIG. 4A) The microRNA can be reversely transcribed into cDNA, which can be then detected with RCA assisted ccs-FISH. FIG. 4B) The sequence of the microRNA is transferred into the immobilized oligo through SplintR ligation, in which the ancillary oligo is ligated with the oligo immobilized on the glass surface. The formed linear DNA is then detected with RCA assisted ccs-FISH.

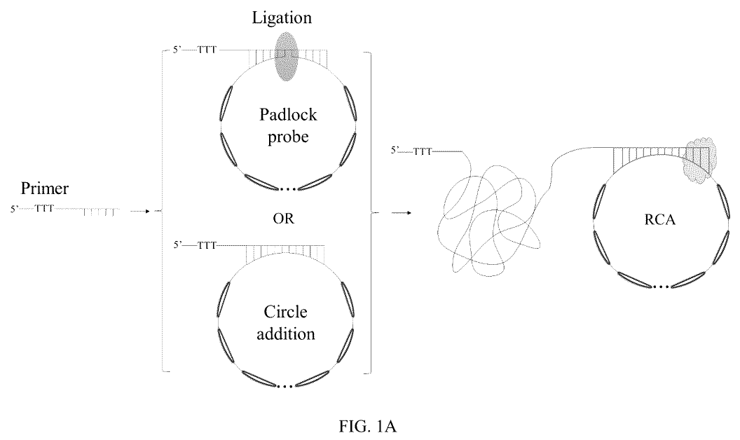

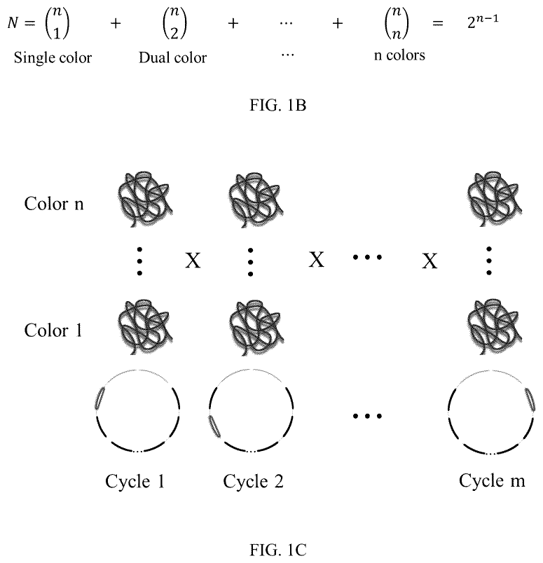

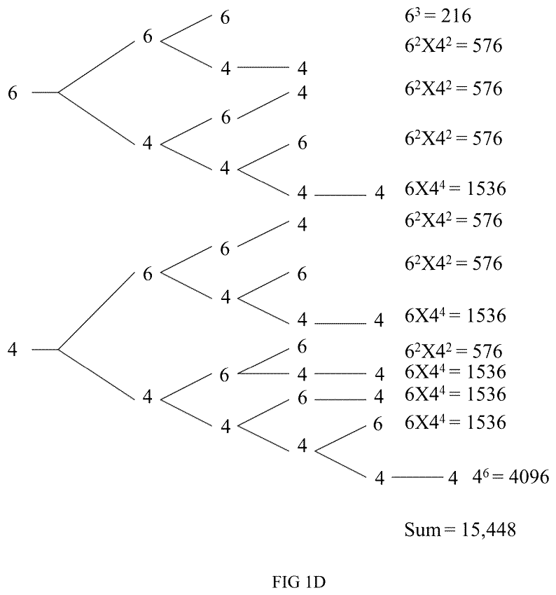

### BRIEF DESCRIPTION OF THE SEQUENCES

SEQ ID NO: 1: exemplary padlock probe sequence
SEQ ID NO: 2: exemplary oligonucleotide primer sequence

### DETAILED DISCLOSURE OF THE INVENTION

As used herein, the singular forms “a”, “an” and “the” are intended to include the plural forms as well, unless the context clearly indicates otherwise. To the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrase “consisting essentially of” or “consists essentially of” indicates that the described embodiment encompasses embodiments containing the specified materials or steps and those that do not materially affect the basic and novel characteristic(s) of the described embodiment.

The term “about” means within an acceptable error range for the particular value as determined by one of ordinary skill in the art, which will depend in part on how the value is measured or determined, i.e., the limitations of the measurement system. In the context of the lengths of polynucleotides where the terms “about” are used, these polynucleotides contain the stated number of bases or base-pairs with a variation of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, such as for the size of the polynucleotides, the combinations and sub-combinations of the ranges (e.g., subranges within the disclosed range) and specific embodiments therein, are explicitly included.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, an “isolated” or “purified” nucleic acid molecule, polynucleotide, antibody, or protein is substantially free of other compounds, such as cellular material, with which it is associated in nature. A purified or isolated polynucleotide (ribonucleic acid (RNA) or deoxyribonucleic acid (DNA)) can be free of the genes or sequences that flank it in its naturally-occurring state. Alternatively, an “isolated” or “purified” nucleic acid molecule or polynucleotide may be RNA or genomic DNA purified from its naturally occurring source, such as a prokaryotic or eukaryotic cell and/or cellular material with which it is associated in nature.

As used herein, “complementary,” in the context of describing two or more polynucleotide sequences, refers to one sequence or subsequences that is at least 60%, 65%, 70%, 75%, 80%, 85%, 90%, 95%, 97%, 98%, or 99% identical to the complement of a second nucleotide sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” or “entirely complementary” refers to a first nucleotide sequence that is 100% identical to the complement of a second nucleic acid.

The term “hybridizes with” or “anneals to” when used with respect to two sequences indicates that the two sequences are sufficiently complementary to each other to allow nucleotide base pairing between the two sequences. Sequences that hybridize or anneal with each other can be perfectly complementary but can also have mismatches to a certain extent. Therefore, the sequences at the 5′ and 3′ ends of the primers described herein may have a few mismatches with the corresponding target sequences at the 5′ and 3′ ends of the target nucleotide sequences as long as the primers can hybridize with the target sequences. Depending upon the stringency of hybridization, a mismatch of up to about 5% to 20% between the two complementary sequences would allow for hybridization between the two sequences. Typically, high stringency conditions have higher temperature and lower salt concentration and low stringency conditions have lower temperature and higher salt concentration. High stringency conditions for hybridization are preferred, and therefore, the sequences at the 3′ and 5′ ends of the primers are preferred to be perfectly complementary to the corresponding target sequences at the 3′ and 5′ ends of the target nucleic acid sequence.

The term “antibody” as used herein refers to a polypeptide encoded by an immunoglobulin gene or immunoglobulin genes, or fragments thereof, which specifically bind and recognize an analyte (antigen). The recognized immunoglobulin light chains are classified as cither kappa or lambda. Immunoglobulin heavy chains are classified as gamma, mu, alpha, delta, or epsilon, which in turn define the immunoglobulin classes, IgG, IgM, IgA, IgD and IgE, respectively. An example of a structural unit of immunoglobulin G (IgG antibody) is a tetramer. Each such tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

Antibodies exist as intact immunoglobulins or as well-characterized fragments produced by digestion of intact immunoglobulins with various peptidases. Thus, for example, pepsin digests an antibody near the disulfide linkages in the hinge region to produce F(ab′)₂, a dimer of Fab which itself is a light chain joined to VH-CH1 by a disulfide bond. The F(ab′)₂ dimer can be reduced under mild conditions to break the disulfide linkage in the hinge region, thereby converting the F(ab′)₂ dimer into two Fab′ monomers. The Fab′ monomer is essentially an Fab with part of the hinge region (see, Paul (Ed.), Fundamental Immunology, Third Edition, Raven Press, N.Y. (1993)). While various antibody fragments are defined in terms of the digestion of an intact antibody, one of skill will appreciate that such fragments may be synthesized de novo either chemically or by utilizing recombinant DNA methodology. Thus, the term “antibody,” as used herein, also includes antibody fragments produced by the modification of whole antibodies.

Antibodies are commonly referred to according their targets. While the nomenclature varies, one of skill in the art will be familiar and understand that several names can be applied to the same antibody. For example, an antibody specific for IgM can be called “anti-IgM,” “IgM antibody,” “anti-IgM antibody,” etc.

The terms “specific for,” “specific to”, “specifically binds,” and grammatically equivalent terms refer to a molecule (e.g., antibody or antibody fragment) that binds to its target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, antibodies that specifically binds a given antibody target will typically bind the antibody target with at least a 2-fold greater affinity than a non-antibody target. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

The term “binds” with respect to an antibody target (e.g., antigen, analyte), typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 75, 80, 85, 90, 91, 92, 93, 94, 95, 96, 97, 98, 99, or 100%). One of skill will recognize that some variability will arise depending on the method and/or threshold of determining binding.

The terms “label,” “detectable label, “detectable moiety,” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, ³²p and other isotopes, haptens, and proteins which can be made detectable, e.g., by incorporating a radiolabel into the peptide or used to detect antibodies specifically reactive with the peptide. The term includes combinations of single labeling agents, e.g., a combination of fluorophores that provides a unique detectable signature, e.g., at a particular wavelength or combination of wavelengths. Any method known in the art for conjugating label to a desired agent may be employed, e.g., using methods described in Hermanson, Bioconjugate Techniques 1996, Academic Press, Inc., San Diego.

In this application, the terms “polypeptide”, “peptide”, and “protein” are used interchangeably herein to refer to a polymer of amino acids. The terms apply to amino acid polymers in which one or more amino acid residues are artificial chemical mimetics of corresponding naturally occurring amino acids, as well as to naturally occurring amino acid polymers and non-naturally occurring amino acid polymers, including those comprising post-translational modifications. As used herein, the terms encompass amino acid chains of any length, including full-length proteins, wherein the amino acid residues are linked by covalent peptide bonds, as well as multi-subunit proteins wherein two or more covalently linked chains of amino acids are associated by covalent bonds or non-covalent interactions.

As used herein, the term “sample” is used in its broadest sense. In some embodiments, a sample is or comprises an animal cell or tissue. In some embodiments, a sample includes a specimen or a culture (e.g., a microbiological culture) obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present technology.

As used herein, the phrases “biological sample” or “sample from a subject” encompasses a variety of sample types obtained from an organism. The term encompasses bodily fluids such as blood, blood components, saliva, nasal mucous, serum, plasma, cerebrospinal fluid (CSF), urine and other liquid samples of biological origin, solid tissue biopsy, tumor, tissue cultures, or supernatant taken from cultured patient cells. The sample can further be obtained from environmental sources. Environmental samples include environmental material such as surface matter, soil, air, water, and industrial samples. The biological sample can be processed prior to assay, such as, for example, washed. The term encompasses samples that have been manipulated after their procurement, such as by treatment with reagents, solubilization, sedimentation, or enrichment for certain components.

As used herein, the term “immobilized” denotes a molecular-based coupling that is not significantly de-coupled (i.e., is irreversible or only reversible on timescales longer than the length of a typical measurement) under the conditions imposed during the steps of the assays described herein. Such immobilization can be achieved through a covalent bond, a non-covalent bond, an ionic bond, an affinity interaction (e.g., avidin-biotin or polyhistidine-Ni⁺⁺), or any other chemical bond.

The term “rolling circle amplification” or “RCA” used herein refers to a nucleic acid amplification reaction in which padlock synthetic probes are used to hybridize a target nucleic acid sequence and create a circle (Nilsson et al., 1994). The circle is then amplified in an RCA reaction.

### Protein Detection

In certain embodiments, a nucleotide padlock probe is provided that possesses a certain number of probe binding sites, wherein the probe binding sites in the padlock probe correspond to sites for the binding of distinct FISH probes that is used to detect a target protein from a sample, including, for example, a biological sample. In certain embodiments, at least 1, 2, 3, 4, 6, 7, 8, 9, 10, about 15, about 20, about 25, about 50, about 100, about 250, about 500, about 1000, about 5000, about 10000, about 50000, about 100000 or more protein or nucleotide targets can be detected. In certain embodiments, the padlock probe further comprises two target recognition sites that bind to an oligonucleotide conjugated to an antibody or a cDNA sequence from the reverse transcription of a microRNA, in which each target recognition site ranges from 10 to 15 bases. In certain embodiments, each probe binding site is a binding site for a fluorescence in situ hybridization (FISH) probe. In certain embodiments, a lock nucleic acid (LNA) modification is adopted in 1, 2, 3, 4, 5, 6, 7, 8, 9, 10, 11, 12, 13, 14, or 15 bases of the FISH probe to match the annealing temperature of the subject FISH probe (8 to 15 bases long) to the normal FISH probe (20 to 30 bases long).

In certain embodiments, TCO-PEG4-NHS functionalizes the primer by a click chemistry between the NHS ester of TCO-PEG4-NHS and the amine group modified at the 5′-end of the primer sequence. In certain embodiments, mTz-PEG4-NHS functionalizes the antibody by a click chemistry between NHS ester of the mTz-PEG4-NHS and the amine on the antibody. In alternative embodiments, the disulfate bond of an antibody can be opened with Tris(2-carboxyethyl)phosphine hydrochloride (TCEP), and a maleimide modified primer sequence (at 5′ end) can react with the exposed —SH bond on the antibody. In alternative embodiments, an antibody can react with a Streptavidin-NHS-ester. In certain embodiments, the NHS ester can react with the amine group on an antibody and conjugate the streptavidin to the antibody. In certain embodiments, a biotinylated oligonucleotide primer can be linked to the antibody through biotin-streptavidin interaction.

In certain embodiments, the primer can be conjugated to an antibody, such as, for example, the B220 antibody, modified with mTz by means of the orthogonal click chemistry between the TCO on the primer sequence and mTz modified on the antibody, specifically, the mTz-PEG4-NHS ester can react with the amine groups on the antibody to modify the mTz to the antibody.

After removing any oligonucleotide sequences not conjugated to the antibody with, for example, Amicon centrifugal filter, a cocktail of primer-antibody conjugate is prepared to contact with the sample containing a target protein. In certain embodiments, unbound antibodies are washed off from the target protein. In certain embodiments, the linear padlock probe anneals to an oligonucleotide primer in a manner such that the 3′ and 5′ end of the linear padlock probe are adjacent to each other, and the 10 to 15 nucleotides at each of the 3′ and 5′ ends of the linear padlock probe anneal to the oligonucleotide primer. In certain embodiments the padlock probe has sequence AACTGTCAGTACATC TCACACCGTC TCACACCGTC TCACACCGTC GTATCATCAA GTATCATCAA GTATCATCAA AGTTCCTAGACTCAA (SEQ ID NO: 1) and the oligonucleotide primer has sequence TTTTTTTTTGATGTACTGACAGTTTTGAGTCTAGGAACT (SEQ ID NO: 2). In certain embodiments, a T4 DNA ligase can be used to circularize the linear padlock probe when the 3′ and 5′ end of the linear padlock probe are adjacent to each other. Alternatively, circular DNA can directly bind to the primer sequence and skip the T4 ligation step. In certain embodiments, a rolling circle amplification in situ is initialized to amplify the circularized padlock probes.

In certain embodiments, a rolling circle amplification reaction is initialized by a DNA polymerase, such as, for example, Phi29 or a Klenow fragment, and the nucleotide primer, which replicates the circularized padlock probe up to thousands of times. In certain embodiments, the replication of the circularized padlock probe creates a single strand of linear DNA up to tens of thousands bases. The resulting long single-stranded, linear DNA coils to form a DNA tangle with a size up to about 500 nm in diameter, which can be detected with next generation sequencing, in situ sequencing, or, preferably, fluorescence in situ hybridization (FISH). In certain embodiments, the DNA tangle comprises a tandem repeat complementary to the padlock probe sequence; it can accommodate thousands of copies of binding sites for each probe.

In certain embodiments, the padlock probe comprises two target recognition sites, each between 10 to 15 bases long, and an intermediate encoding region up to 230 bases, corresponding to up to 30 binding sites for FISH probes from 8 to 15 bases long. In certain embodiments, the target recognition sites are sites at which an oligonucleotide primer sequence conjugated to an antibody or a cDNA sequence derived from a microRNA can bind. In certain embodiments, the DNA tangle is stained in a binding site by binding site fashion, in which at least one of the probe binding sites is detected in every cycle and, after imaging, the FISH probes are stripped off with, for example, formamide buffer. Therefore, every DNA tangle will generate a barcode after certain number imaging cycles, in which each cycle uses a distinct set of different types of FISH probes. In certain embodiments, in every cycle, each DNA tangle is stained with a certain color or a combination of colors, and after a FISH probe removal, the DNA tangles are incubated with another set of FISH probes and stained with another color or combination of colors. In certain embodiments, every DNA tangle will generate a barcode after a certain number cycles of staining and probe removal.

In certain embodiments, the DNA tangle can be detected with fluorescence in situ hybridization with multiplexity achieved through sequential FISH. All DNA tangles are stained in a binding site by binding site fashion, in which one of the probe binding sites is detected in every cycle and after imaging the FISH probes are stripped off with formamide buffer. In certain embodiments, the detection of the DNA tangle can further comprise of a combination of 2, 3, 4, 5, . . . , to m FISH probes, where m is the number single colors available in a single cycle, with the color combinatorial FISH (cc-FISH). Based on the design, 2^{m}−1 types of DNA tangles can be detected with a m-color combination in a single round of imaging when 1, 2, . . . , to m colors are selected to represent a single DNA tangle, where m is the number of single colors available in a single cycle. The cc-FISH not only breaks the limitation of spectral overlap, but also reduces the amount of storage volume required for recording targets by more than half.

In certain embodiments, after the RCA, a pool of DNA tangles are formed, which are then detected with ccs-FISH. In certain embodiments, at least 1, 2, 3, 4, 5, 6, 7, 8, or more types of FISH probes, in which each FISH probe has a distinct fluorophore, can be incubated with the DNA tangles. In certain embodiments, each DNA tangle will allow at least 1, 2, 3, 4, 5, 6, 7, 8, 9, 10, 11, 12, . . . , up to 30 types of FISH probes to bind. For those DNA tangles that only have one type of probe binding, the fluorophore of the FISH probe is visualized as single color spot using, for example, fluorescence microscopy. For those DNA tangles who have two or more colors binding to them will appear as a mixed color as a single spot under fluorescence microscopy. Therefore, each DNA tangle can have a color from the pallet of 2″-1 colors made by the n type of FISH probes of different colors.

The image is processed with convolutional neural network model to resolve the color code for each DNA tangle. In certain embodiments, after imaging, all probes will be flushed away with washing buffer.

In certain embodiments, in the next cycle, the process is repeated with at least 1, 2, 3, 4, 5, 6, 7, 8, or more types of FISH probes, we get another color for each DNA tangle. After certain number of cycles, such as, for example, about 2 to about 30 cycles, all images will be registered and every DNA tangle will get a color barcode of different length. In certain embodiments, more than one FISH probe may bind to a single DNA tangle, with each probe occupying one probe binding site. Whereas, some DNA tangles are only stained with only one color, therefore, only consume one probe binding sites. The overall number of probe binding sites are the same for all DNA tangles. Therefore, some DNA tangles will use up their probe binding sites very quickly if they are stained with multiple colors in every single cycle, causing a short barcode. In certain embodiments, the target molecules based on these digital codes.

In certain embodiments, the DNA tangle can be detected with the color combinatorial and sequential FISH (ccs-FISH). All DNA tangles are stained with n types of FISH probes of different colors, where n is the number of colors available in a single cycle. In a single cycle, a multiplexity of 2ⁿ-1 can be achieved. In the first cycle, a DNA tangle stained with k colors (k≤n) will consume k binding sites for FISH probe. In the next cycle, the ccFISH is performed over the remaining FISH probe binding sites. The number of the multiplexity can be calculated with exhaustive enumeration. For example, when 4 colors are used to encode the DNA tangles possessing 6 probe binding sites, with each DNA tangle stained with only one or two colors, the multiplexity can be 15,448.

For example, one can summarize as follows:

1. If we are doing seq-FISH, all the m types of FISH probes will bind to the first FISH probe binding sites of all DNA tangles, but not the 1st to m^{th} FISH probe binding sites of a single DNA tangle. After imaging the first cycle, the m types of FISH probes will be removed. Another m types of FISH probes will bind to the 2nd FISH probe binding sites of all DNA tangles. After imaging the 2nd cycle, the m types of probes will be removed. We repeat n cycles, giving us a multiplexity of m″.

2. In the cc-FISH:

The m types of FISH probes can bind to any number of the first m^{th} FISH probe binding sites of an individual DNA tangle, depending on how many colors are used to represent this DNA tangle. For example, if the DNA tangle is encoded with a single color, only the first FISH probe binding sites will be occupied. If the DNA tangle is encoded with two colors, only the first two FISH probe binding sites will be occupied. . . . , if the DNA tangle is encoded with m colors, the first m^{th} FISH probe binding sites will be occupied. This gives us a multiplexity of 2^{m}-1.

3. In the ccs-FISH, the ccFISH described above in point 2 happens in first cycle. After that, we remove the m types of FISH probes and apply another m types of FISH probes, each with different color, to the DNA tangle. The m types of FISH probes will do another round of cc-FISH to the remaining binding sites on the DNA tangle. This process will move on, until the FISH probe binding sites are used up in later cycles. Since different number of FISH probe binding sites will be occupied in each cycle for each DNA tangle, the FISH probe binding sites of some DNA tangles will be used up in short cycles, causing short barcode. Whereas some of the DNA tangles will use up their FISH probe binding sites to the last cycle (the maximum number of FISH probe binding sites on a DNA padlock probe).

In certain embodiments, the staining and removal of the probes during the sequential FISH (seq-FISH) can be performed by flushing the probe mix at high concentration and prewarmed formamide-based or dimethyl sulfoxide-based washing buffer through the RCA amplicons.

The presently described assays involve the use of a solid support, typically a glass chamber wall. For detection of the FISH probes by fluorescent microscopy or confocal microscopy, a solid support that emit high levels of autofluorescence should be avoided since this will increase background signal and potentially render them unsuitable.

In certain embodiments, physical parameters can be used as differentiation parameters to distinguish the probes excitable fluorescent dyes or colored dyes that impart different emission spectra and/or scattering characteristics to the probes. Alternatively, different concentrations of combinations of one or more fluorescent dyes can be used for distinguishing or differentiating probes.

When the distinguishable characteristic is a fluorescent dye or color, it can be bound to the molecules of the probe. Probes with dyes already incorporated and thereby suitable for use in the present disclosure are commercially available, from suppliers, such as, for example, Spherotech, Inc. (Libertyville, Ill., USA) and Molecular Probes, Inc. (Eugene, Oreg., USA).

Labels can be any substance or component that directly or indirectly emits or generates a detectable signal. In some embodiments, the labels are fluorophores, many of which are reported in the literature and thus known to those skilled in the art, and many of which are readily commercially available. Literature sources for fluorophores include Cardullo et al., Proc. Natl. Acad. Sci. USA 85: 8790-8794 (1988); Dexter, J. of Chemical Physics 21: 836-850 (1953); Hochstrasser et al., Biophysical Chemistry 45: 133-141 (1992); Selvin, Methods in Enzymology 246: 300-334 (1995); Steinberg, Ann. Rev. Biochem., 40: 83-114 (1971); Stryer, Ann. Rev. Biochem. 47: 819-846 (1978); Wang et al., Tetrahedron Letters 31: 6493-6496 (1990); and Wang et al., Anal. Chem. 67: 1197-1203 (1995). The following are non-limiting examples of fluorophores that can be used as labels:

4-acetamido-4′-isothiocyanatostilbene-2,2′disulfonic acid; acridine; acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin; 7-amino-4-methylcoumarin (AMC, Coumarin 120); 7-amino-4-trifluoromethylcoumarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-(4′-dimethylaminophenylazo)benzoic acid (DABCYL); 4-dimethylaminophenylazophenyl-4′-isothiocyanate (DABITC); cosin; cosin isothiocyanate; erythrosin B; erythrosin isothiocyanate; ethidium; 5-carboxyfluorescein (FAM); 5-(4,6-dichlorotriazin-2-yl)aminofluorescein (DTAF); 2′, 7′-dimethoxy-4′5′-dichloro-6-carboxyfluorescein (JOE); fluorescein; fluorescein isothiocyanate; fluorescamine; IR144; IR1446; Malachite Green isothiocyanate; 4-methylumbelliferone; ortho cresolphthalein; nitrotyrosine; pararosaniline; Phenol Red; phycoerythrin ((PE) including but not limited to B and R types); o-phthaldialdehyde; pyrene; pyrene butyrate; succinimidyl 1-pyrene butyrate; quantum dots; Reactive Red 4 (Cibacron™ Brilliant Red 3B-A); 6-carboxy-X-rhodamine (ROX); 6-carboxyrhodamine (R6G); lissamine rhodamine B sulfonyl chloride rhodamine; rhodamine B; rhodamine 123; rhodamine X isothiocyanate; sulforhodamine B; sulforhodamine 101; sulfonyl chloride derivative of sulforhodamine 101 (Texas Red); N,N,N′, N′-tetramethyl-6-carboxyrhodamine (TAMRA); tetramethyl rhodamine; tetramethyl rhodamine isothiocyanate (TRITC); riboflavin; rosolic acid; and lanthanide chelate derivatives.

Particular fluorophores for use in the disclosed immunoassays include fluorescein, fluorescein isothiocyanate, phycoerythrin (PE), rhodamine B, and Texas Red (sulfonyl chloride derivative of sulforhodamine 101). Any of the fluorophores in the list preceding this paragraph can be used in the presently described assays to label the probe. Fluorochromes can be attached by conventional covalent bonding, using appropriate functional groups on the fluorophores and on the probe. The recognition of such groups and the reactions to form the linkages will be readily apparent to those skilled in the art.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1—Spatial Protein Profiling

The RCA-based ccs-FISH is a highly multiplexed detection technology with quantitative capability by enumeration. The detection target includes both nucleic acid and protein.

The RCA-assisted ccs-FISH is advantageous when applied for the spatial protein profiling over the state-of-the-art technology for protein detection in situ in the following aspects: 1) signal amplification compatible with different tissue preparations and auto-fluorescent tissues; 2) all-in-one staining, which incubates all target proteins with a cocktail of antibodies; 3) color combination to break the limit of spectral overlap by encoding ten types of target proteins with only four colors; 4) experimental simplicity with a minimum number cycles; 5) low storage volume required for recording the detected proteins.

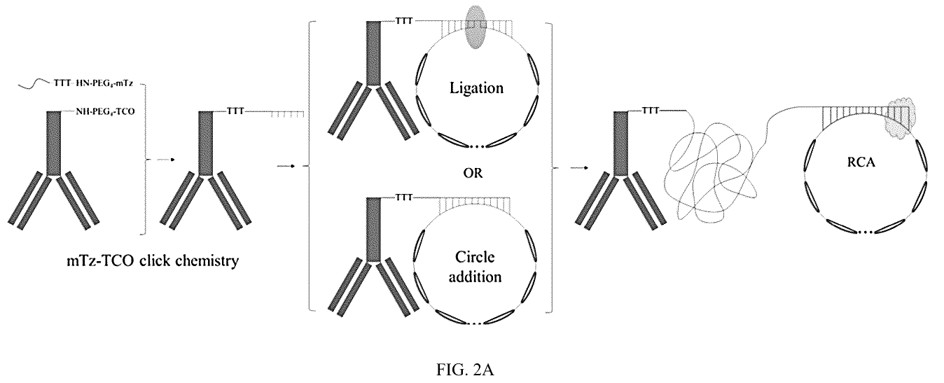

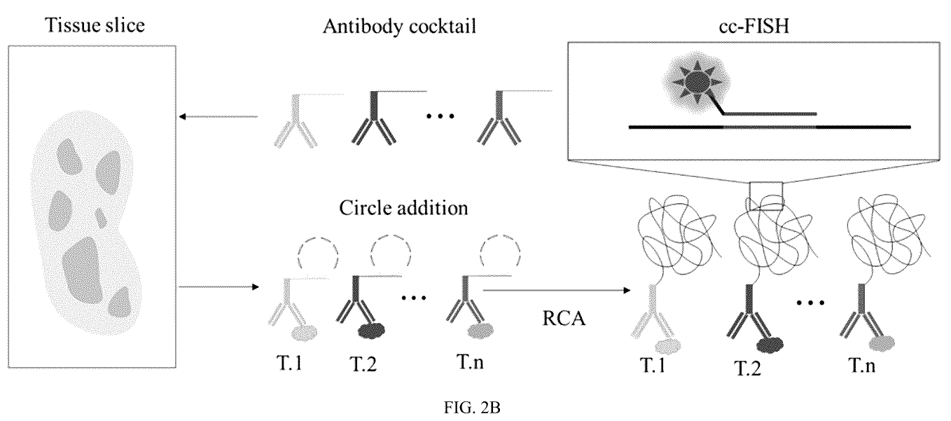

FIGS. 2A-2B show the workflow in the application of RCA-assisted ccs-FISH to the spatial protein profiling. TCO-PEG4-NHS functionalizes the primer by a click chemistry between the NHS ester of TCO-PEG4-NHS and the amine group modified at the 5′-end of the primer sequence. mTz-PEG4-NHS functionalizes the antibody by a click chemistry between NHS ester of the mTz-PEG4-NHS and the amine on the antibody. Then, the primer can be conjugated to an antibody modified with mTz by means of the orthogonal click chemistry between the TCO on the primer sequence and mTz modified on the antibody. After removing the redundant oligo complex with, for example, Amicon centrifugal filter, a cocktail of primer-antibody conjugate is prepared to contact with the sample containing a target protein. After washing off unbound antibodies from the sample, the linear padlock probe anneals to a nucleotide primer in a manner such that the 3′ and 5′ end of the linear padlock probe are adjacent to each other, and the 10 to 15 nucleotides at each of the 3′ and 5′ ends of the linear padlock probe anneal to the primer. The T4 DNA ligase can be used to circularize the linear padlock probe when the 3′ and 5′ end of the linear padlock probe are adjacent to each other. Alternatively, circular DNA can directly bind to the primer sequence and skip the T4 ligation step. Finally, a rolling circle amplification in situ is initialized to amplify the circularized padlock probes and the DNA tangles can be detected with ccFISH.

### Example 2—Protein Quantification with a Single Drop of Blood

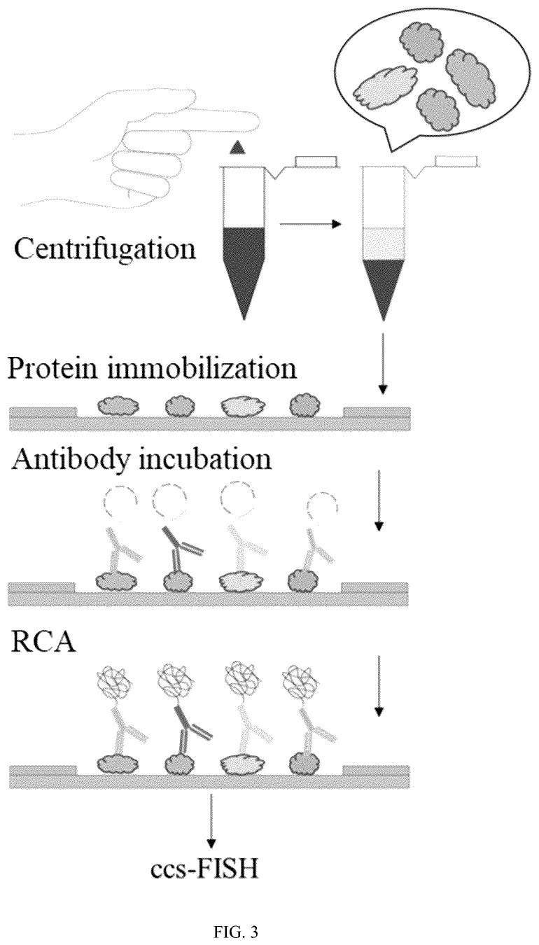

The highly multiplexed quantification of protein markers is of great value in the diagnosis of various diseases. Even though mass spectrometry can profile the protein content in the plasma, the sample preparation is cumbersome and requires large amounts of blood. The quantification of proteins through digital ELISA can only detect a single type of protein, which is far from enough to give informational diagnosis and prognosis. The RCA-assisted immuno-ccs-FISH possesses massive multiplexing capability for high-dimensional protein quantification. One drop of blood can provide sufficient number of proteins for the analysis. FIG. 3 is the schematic of the RCA-assisted ccs-FISH applied to the protein profiling of the plasma. The cellular content and vesicles are removed by centrifugation, and the supernatant protein can be biotinylated by the click chemistry between sulfo-NHS-biotin and amine groups on the proteins. After the biotinylated proteins are immobilized onto the glass chamber modified with streptavidin, the antibody cocktail, as shown in FIG. 2B, can be added into the chamber to target the proteins of interest. Next, the RCA in situ will synthesis a DNA tangle anchored to the protein of interest, which can be enumerated with the ccs-FISH.

### Example 3—MicroRNA Profiling

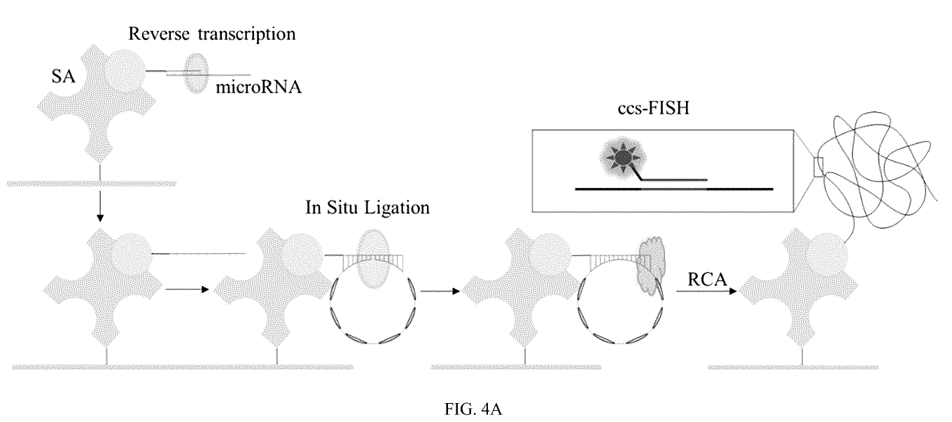

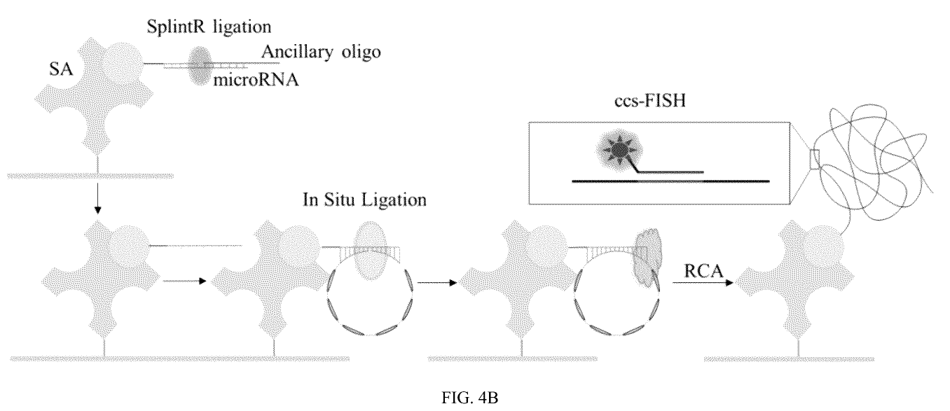

The RCA-assisted ccs-FISH can also be used for microRNA profiling. The microRNAs can be reversely transcribed into cDNAs when captured by the primer sequences immobilized on the glass chamber through covalent bond or biotin/streptavidin interaction. Once the microRNA content is digested by RNase H, the cDNA sequences serve as the ligation and amplification primer. After the sequence specific padlock probe anneal to the cDNA sequences, a ligation in situ will circularize the padlock probe, which is then amplified into DNA tangles with RCA. Finally, different types of microRNAs can be enumerated with the ccs-FISH, as shown in FIG. 4.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method of protein detection, the method comprising:
a) providing a linear DNA padlock probe annealed to an oligonucleotide primer sequence conjugated to an antibody or a circular DNA probe annealed to an oligonucleotide primer sequence conjugated to an antibody, wherein the linear DNA padlock probe comprises 2 to 30 fluorescence in situ hybridization (FISH) probe binding sites and 2 target recognition sites and wherein the circular DNA probe comprises 2 to 30 fluorescence in situ hybridization (FISH) probe binding sites and 1 target recognition site;
b) providing a sample containing a target protein;
c) contacting the oligonucleotide primer conjugated to the antibody to the target protein, wherein the target protein and the antibody bind together;
d) circularizing the linear DNA padlock probe annealed to the oligonucleotide primer sequence;
e) amplifying the circularized DNA padlock probe or the circular DNA probe to yield a single strand of linear DNA; and
f) contacting a first FISH probe and a second FISH probe, to the single strand of linear DNA and detecting the first and second FISH probes.

2. The method of claim 1, wherein the amplification of step e) is rolling circle amplification.

3. The method of claim 1, wherein the oligonucleotide primer sequence binds to the 2 target recognition sites to initiate the circularization of the linear DNA padlock probe of step d).

4. The method of claim 1, wherein each of the 1 or 2 target recognition sites is about 10 to about 15 bases long.

5. The method of claim 1, wherein the circularization of the linear DNA padlock probe initiates the amplification of step e).

6. The method of claim 1, wherein each of the 2 to 30 FISH probe binding sites is about 8 to about 15 bases long.

7. The method of claim 1, wherein each of the first FISH probe and the second FISH probe has a distinct fluorophore.

8. The method of claim 1, wherein the contacting of the first FISH probe and the second FISH probe occurs simultaneously.

9. The method of claim 1, wherein step f) comprises:
i) contacting the first FISH probe to the single strand of linear DNA;
ii) imaging the single strand of linear DNA;
iii) contacting the second FISH probe to the single strand of linear DNA;
iv) imaging the single strand of linear DNA; and
v) overlaying the images resulting from step ii) and step iv).

10. The method of claim 1, wherein step f) comprises:
i) contacting the first FISH probe and the second FISH probe to the single strand of linear DNA;
ii) imaging the single strand of linear DNA specific with a color of the first FISH probe;
iii) imaging the single strand of linear DNA specific with a color of the second FISH probe; and
iv) overlaying the images resulting from step ii) and step iii).

11. A method of microRNA detection, the method comprising:
a) providing a linear DNA padlock probe annealed to a cDNA sequence reversely transcribed from a microRNA sequence or a circular DNA probe annealed to a cDNA sequence reversely transcribed from a microRNA sequence, wherein the linear DNA padlock probe comprises 2 to 30 fluorescence in situ hybridization (FISH) probe binding sites and 2 target recognition sites and wherein the circular DNA probe comprises 2 to 30 fluorescence in situ hybridization (FISH) probe binding sites and 1 target recognition site;
b) circularizing the linear DNA padlock probe annealed to the cDNA sequence reversely transcribed from a microRNA sequence
c) amplifying the circularized DNA padlock probe or the circular DNA probe to yield a single strand of linear DNA; and
d) contacting a first FISH probe and a second FISH probe to the single strand of linear DNA and detecting the first and second FISH probes.


SEQUENCE LISTING (2 sequences)

SEQ ID NO: 1 - DNA, 90 bp, synthetic construct
  source 1..90: mol_type = other DNA
aactgtcagtacatctcacaccgtctcacaccgtctcacaccgtcgtatcatcaagtatcatcaagtatcatcaaagttcctagactcaa

SEQ ID NO: 2 - DNA, 39 bp, synthetic construct
  source 1..39: mol_type = other DNA
tttttttttgatgtactgacagttttgagtctaggaact